(19) United States
(12) Patent Application Publication
(10) Pub. No.: US 2019/0175262 A1
(43) Pub. Date: Jun. 13, 2019

(54) BALLOON CATHETER DISTAL END COMPRISING ELECTRODES AND THERMOCOUPLES

(71) Applicant: BIOSENSE WEBSTER (ISRAEL) LTD., Yokneam (IL)

(72) Inventors: ASSAF GOVARI, Haifa (IL); Christopher Thomas Beeckler, Brea, CA (US); Andres Claudio Altmann, Haifa (IL)

(21) Appl. No.: 15/837,339

(22) Filed: Dec. 11, 2017

(51) Int. Cl. A61B 18/14; A61M 25/10; A61M 25/00

(52) U.S. Cl. CPC A61B 18/1492; A61B 2017/00092; A61M 25/0009; A61M 25/1027

(57) ABSTRACT

A method for producing a medical instrument, the method includes coupling a balloon-based distal end of the medical instrument to a jig that sets the distal end to an expanded position. While the distal end is coupled to the jig, one or more electrodes are disposed on an outer surface of the distal end, one or more openings are formed in a wall of the distal end, and are threaded through the openings respective leads coupled to at least one of respective sensors and electrodes that are mounted on the outer surface of the distal end. One or more patches that cover the openings and couple the at least one of respective sensors and electrodes to the outer surface of the distal end, are coupled on the outer surface of the distal end.

### FIELD OF THE INVENTION

The present invention relates generally to catheters, and particularly to balloon catheters and methods and systems for producing balloon catheters.

### BACKGROUND OF THE INVENTION

Balloon catheters may be used in various medical procedures, such as in cardiac ablation. Several techniques for producing balloon catheters are known in the art.

For example, U.S. Pat. No. 6,500,174 describes a medical balloon catheter assembly that includes a balloon having a permeable region and a non-permeable region. The balloon is constructed at least in part from a fluid permeable tube such that the permeable region is formed from a porous material, which allows a volume of pressurized fluid to pass from within a chamber formed by the balloon and into the permeable region sufficiently such that the fluid may be ablatively coupled to tissue engaged by the permeable region.

U.S. Pat. No. 5,865,801 describes a balloon catheter that includes an elongate pliable catheter tubing with a dilatation balloon fixed to the catheter tubing near its distal end. The dilatation balloon includes a first wall for dividing the balloon into a plurality of dilatation compartments adjacent one another and arranged angularly about the catheter tubing.

U.S. Pat. No. 5,275,597 describes a catheter combination using a percutaneous transluminal transmitter for transmitting energy to a localized area. The combination includes a catheter having a hollow tubular member. A transmitter combination for partial insertion into the catheter includes a continuous central conductor terminating in a tip for receiving and transmitting a signal to the tip.

### SUMMARY OF THE INVENTION

An embodiment of the present invention that is described herein provides a method for producing a medical instrument, the method includes coupling a balloon-based distal end of the medical instrument to a jig that sets the distal end to an expanded position. While the distal end is coupled to the jig, one or more electrodes are disposed on an outer surface of the distal end, one or more openings are formed in a wall of the distal end, and are threaded through the openings respective leads coupled to at least one of respective sensors and electrodes that are mounted on the outer surface of the distal end. One or more patches that cover the openings and couple the at least one of respective sensors and electrodes to the outer surface of the distal end, are coupled on the outer surface of the distal end.

In some embodiments, forming the openings includes cutting a latitudinal opening in the wall of the balloon-based distal end. In other embodiments, the sensors include one or more thermocouples (TCs). In yet other embodiments, coupling the balloon-based distal end to the jig includes inserting the balloon-based distal end into a hollow templates having one or more patterned openings.

In an embodiment, depositing the electrodes includes sputtering atoms or ions through the patterned openings. In another embodiment, sputtering the atoms or ions includes impinging electrons or ions on a sputtering target. In yet another embodiment, the method includes, before depositing the electrodes through the patterned openings, attaching the outer surface of the balloon-based distal ends to an inner surface of the hollow template, by creating vacuum around the balloon-based distal ends.

In some embodiments, the balloon-based distal end includes an inflatable balloon made from polyethylene terephthalate (PET). In other embodiments, the balloon-based distal end includes an inflatable balloon made from polyurethane. In yet other embodiments, the balloon-based distal end includes an inflatable balloon made from polyether block amide.

In an embodiment, coupling the one or more patches that cover the openings includes sealing the openings. In another embodiment, coupling the one or more patches includes cementing the at least one of respective sensors and electrodes to the outer surface of the distal end.

In some embodiments, the electrodes include one or more ablation electrodes. In other embodiments, the sensors include one or more electrophysiology (EP) sensing electrodes.

The present invention will be more fully understood from the following detailed description of the embodiments thereof, taken together with the drawings in which:

### BRIEF DESCRIPTION OF THE DRAWINGS

FIG. 1 is a schematic, pictorial illustration of a catheter-based tracking and ablation system, in accordance with an embodiment of the present invention;

FIG. 2 is a schematic, pictorial illustration of a balloon assembly, in accordance with an embodiment of the present invention;

FIG. 3 is a flow chart that schematically illustrates a method for producing a balloon assembly of a catheter distal end, in accordance with an embodiment of the present invention; and

FIG. 4 is a schematic, sectional view of a balloon assembly contained within a production jig, in accordance with an embodiment of the present invention.

### DETAILED DESCRIPTION OF EMBODIMENTS

### Overview

Balloon catheters are used, for example, in various interventional cardiology procedures, such as in treating arrhythmia, by ablating tissue so as to form a lesion that blocks electrical conduction along a path of the tissue in a patient heart. A lesion that blocks undesired intra-heart electrical signals may be formed using various techniques, such as by electrophysiology (EP) mapping of the tissue, followed by applying a radio-frequency (RF) ablation to the tissue at one or more selected locations. In principle, monitoring the ablation process can be carried out using sensors mounted on the balloon catheter.

A catheter used for ablation may comprise an inflatable balloon assembly having an array of devices, such as ablation electrodes and sensors, mounted on an outer surface of the balloon assembly. The electrodes and sensors typically exchange electrical signals with a proximal end of the balloon catheter, via electrical leads. In some cases, such balloon assemblies have no openings via which the electrical leads can be connected to the devices mounted on the outer surface of the balloon assembly.

Embodiments of the present invention that are described hereinbelow provide improved techniques for depositing electrodes and/or mounting sensors of various types, such as thermocouples (TCs), on an outer surface of a balloon-based distal end of a catheter. These techniques are further used for electrically connecting the electrodes and/or sensors to the proximal end of the catheter using a single production setup.

In some embodiments, during production, the balloon-based distal end is coupled to a jig, which is configured to set the distal end to an expanded position. The following process steps are carried out while the distal end is coupled to the jig:
Electrodes are deposited on using sputtering process, and one or more TCs are mounted on the outer surface of a wall of the distal end.
One or more openings are formed at given locations of the wall of the distal end, and electrical leads that typically extend from the catheter proximal end, are threaded through the openings and electrically coupled to the electrodes and/or TCs.
One or more patches are coupled to the given locations on the outer surface of the distal end so as to cover the respective openings and to couple the respective TCs to the outer surface of the balloon-based distal end.

In the context of the present disclosure and in the claims, the terms “balloon,” “balloon-based distal end” and “balloon assembly” are used interchangeably and refer to any suitable medical balloon catheter.

Medial catheters produced using the disclosed techniques are highly functional, due to the sputtering of high quality electrodes on the balloon catheter. The disclosed techniques enable seamless integration of various sensors and respective leads into the catheters.

Furthermore, the disclosed techniques reduce the production cost of the balloon catheter because multiple process steps are applied with the distal end coupled to a jig.

### System Description

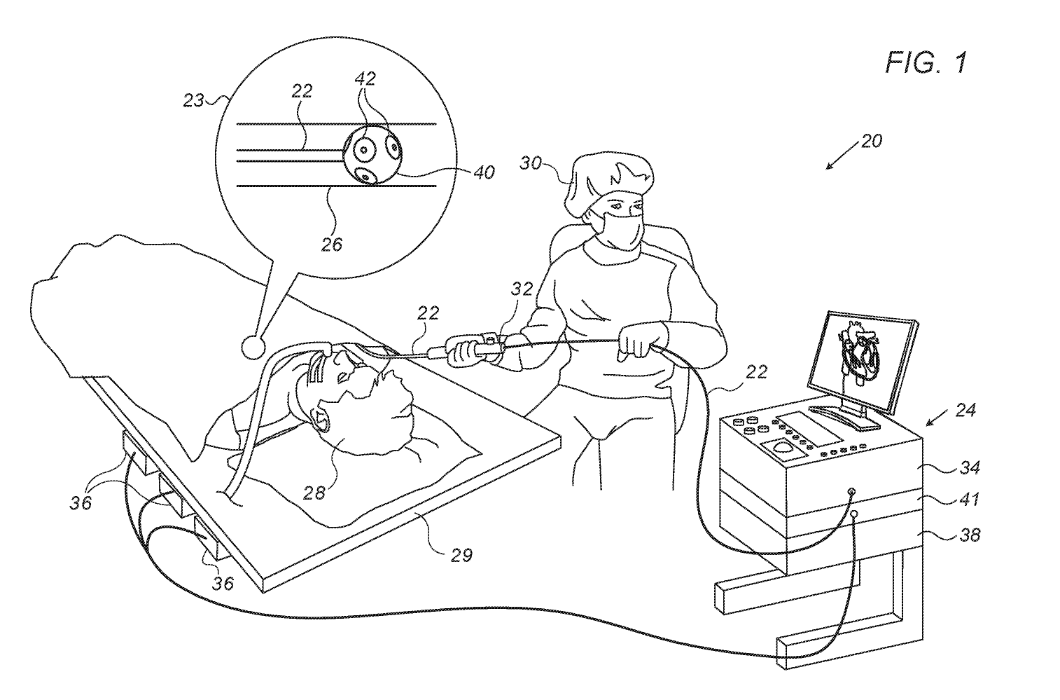

FIG. 1 is a schematic, pictorial illustration of a catheter-based tracking and ablation system 20, in accordance with an embodiment of the present invention. System 20 comprises a catheter 22, in the present example a cardiac catheter, and a control console 24. In the embodiment described herein, catheter 22 may be used for any suitable therapeutic and/or diagnostic purposes, such as ablation of tissue in a heart (not shown).

Console 24 comprises a processor 34, typically a general-purpose computer, with suitable front end and interface circuits 38 for receiving signals via catheter 22 and for controlling the other components of system 20 described herein.

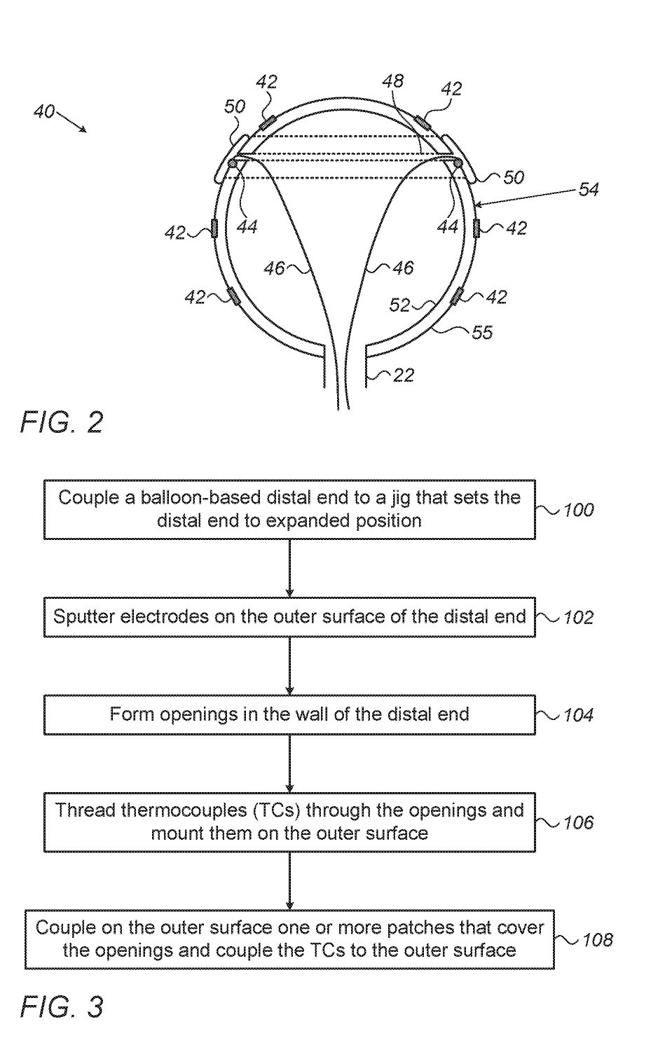

Reference is now made to an inset 23. A physician 30 inserts a medical instrument, such as catheter 22, through a blood vessel 26 of the vascular system of a patient 28 lying on a table 29. Catheter 22 comprises a balloon-based distal end assembly, such as a balloon assembly 40 fitted at its distal end. In some embodiments, assembly 40 comprises an inflatable balloon having a wall (shown in FIG. 2 below) made from polyethylene terephthalate (PET), or from polyurethane, or from a thermoplastic elastomer, such as polyether block amide, or from any other suitable flexible material. In some embodiments, balloon assembly 40 comprise electrodes 42 that may be used for multiple purposes, such as electrophysiology (EP) mapping of tissue, or for ablating tissue at a target location of the heart.

In some embodiments, ablation electrodes 42 are deposited on the outer surface of balloon assembly 40 using a suitable geometrical pattern that fits the shape of the organ in question and the corresponding medical procedure (e.g., EP mapping, tissue ablation).

Several techniques may be used for applying the deposition, such as sputtering techniques, as will be described in detail in relation to FIG. 2 3 below.

In some embodiments, balloon assembly 40 may comprise one or more sensors, such as thermocouples (TCs) (shown in FIG. 2 below) configured to measure tissue temperature, so as to monitor the ablation procedure.

In other embodiments, balloon assembly 40 may comprise any additional or alternative suitable kinds of sensors, such as electrodes used for EP mapping tissue in the heart of patient 28.

During the insertion of catheter 22, balloon assembly 40 is contained in a sheath (not shown) in a collapsed position. In an embodiment, physician 30 navigates balloon assembly 40 in the vicinity of the target location in the heart by manipulating catheter 22 with a manipulator 32 near the proximal end of the catheter. The proximal end of catheter 22 is connected to interface circuitry in processor 34.

In an embodiment, after navigating assembly 40 to the target location, physician 30 may inflate balloon assembly 40 so as to make physical contact between electrodes 42 and tissue at the target location. In an embodiment, electrodes 42 are configured to receive electrical ablation signals, such as radio-frequency (RF), via suitable wires that run through catheter 22, and to ablate tissue at the target location in the patient heart.

As noted above, the temperature of the ablation procedure may be monitored using the TCs of assembly 40. The ablation procedure is typically carried out at a predefined temperature range so as to enable the formation of a desired lesion without causing heart damage that may risk the safety of patient 28.

In some embodiments, the position of balloon assembly 40 in the heart cavity is measured by a position sensor (not shown) of a magnetic position tracking system. In this case, console 24 comprises a driver circuit 41, which drives magnetic field generators 36 placed at known positions external to patient 28 lying on table 29, e.g., below the patient's torso. The position sensor is configured to generate position signals in response to sensed external magnetic fields from field generators 36. The position signals are indicative of the position of balloon assembly 40 in the coordinate system of the position tracking system.

This method of position sensing is implemented in various medical applications, for example, in the CARTO™ system, produced by Biosense Webster Inc. (Irvine, Calif.) and is described in detail in U.S. Pat. Nos. 5,391,199, 6,690,963, 6,484,118, 6,239,724, 6,618,612 and 6,332,089, in PCT Patent Publication WO 96/05768, and in U.S. Patent Application Publications 2002/0065455 A1, 2003/0120150 A1 and 2004/0068178 A1, whose disclosures are all incorporated herein by reference.

Processor 34 typically comprises a general-purpose computer, which is programmed in software to carry out the functions described herein. The software may be downloaded to the computer in electronic form, over a network, for example, or it may, alternatively or additionally, be provided and/or stored on non-transitory tangible media, such as magnetic, optical, or electronic memory.

### Integrating Thrmocouples in a Balloon Catheter

FIG. 2 is a schematic, pictorial illustration of balloon assembly 40, in accordance with an embodiment of the present invention. In some embodiments, balloon assembly 40 comprises a wall 54 having an inner surface 52 and an outer surface 55, which are coupled to the distal end of catheter 22.

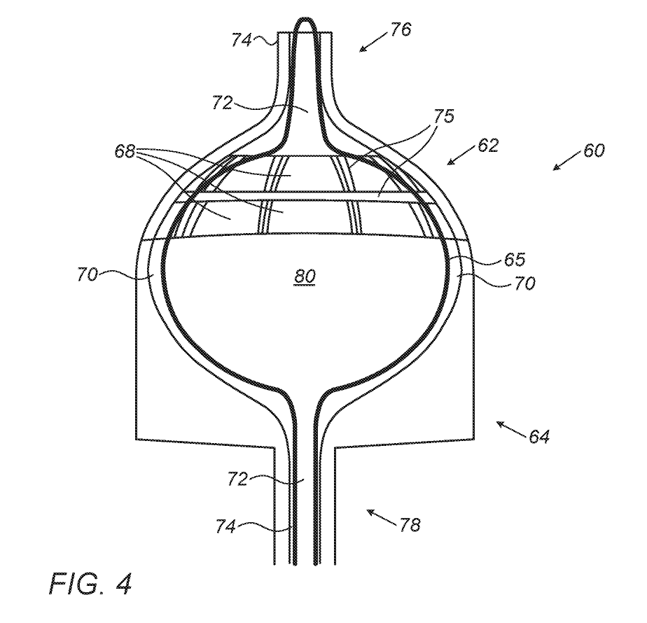

In some embodiments, balloon assembly 40 comprises electrodes 42 deposited on outer surface 55 using, for example, a sputtering process. In some embodiments, during production, balloon assembly 40 may be coupled to a jig (shown in FIG. 4 below), which is configured to set balloon assembly 40 to an expanded position during the sputtering process. The production process of balloon assembly 40 is described in detail in FIG. 3 below.

In some embodiments, balloon assembly 40 further comprises one or more TCs 44 mounted on outer surface 55 of assembly 40. In some embodiments, each TC 44 is configured to sense the temperature of tissue in contact with outer surface 55, and to produce electrical signals indicative of the sensed temperature.

In some embodiments, each TC 44 is coupled to a respective electrical lead 46 comprising two electrically isolated wires, which extends through the internal volume of balloon assembly 40, via catheter 22, to processor 34 or to any suitable interface of console 24. In some embodiments, leads 46 are configured to conduct the electrical signals between TC 44 and processor 34.

In some embodiments, leads 46 may be arranged in any suitable configuration. In an embodiment, each lead 46 may extend directly between TC 44 and processor 34, such that multiple leads 46 may be arranged, for example, in a braid within catheter 22.

In an alternative embodiment, lead 46 may be electrically connected, e.g., via a connector located at the distal end of catheter 22, to a common electrical wire that extends between the connector and processor 34.

In some embodiments, balloon assembly 40 comprises one or more openings 48. In some embodiments, in producing the balloon, opening 48 may be formed by one or more horizontal cuts on respective latitudes along the circumference of wall 54 of balloon assembly 40, as shown in FIG. 2. In this configuration, some or all of TCs 44 are threaded at the same latitudinal of balloon assembly 40.

In other embodiments, any other suitable configuration of the openings may be applied. For example, the openings may comprise roundly-shaped holes at specific locations of wall 54. Alternatively or additionally, vertical cuts along one or more longitudes of wall 54 may be used to form the openings in balloon assembly 40.

In some embodiments, openings 48 are formed and leads 46 are threaded through openings 48, while balloon assembly 40 is still coupled to the jig (e.g., before or after depositing electrodes 42).

In some embodiments, leads 46 are threaded through openings 48 such that each TC 44 is mounted on outer surface in close proximity to opening 48. In alternative embodiments, TC 44 are mounted not necessarily with close proximity to openings 48.

In some embodiments, balloon assembly 40 further comprises one or more patches 50, which are configured to adhere to outer surface 55, using glue material or using any other suitable technique.

In some embodiments, each patch 50 is adapted to cover a respective opening 48 so as to prevent unintended flow of fluids (e.g., blood and/or irrigation fluid) through openings 48, between the internal volume of assembly 40 and the body of patient 28. This configuration enables inflation (e.g., using a saline solution) and deflation of balloon assembly 40 in a controlled manner, typically through proximal end 32.

In some embodiments, after creating patches 50, and electrodes 42, irrigation holes (not shown) can be made in the balloon allowing for an irrigation path into the patient.

In some embodiments, each patch 50 is further configured to couple a respective TC 44 to outer surface 55 at a predefined location. In some embodiments, a single patch 50 may be coupled to outer surface 55 along a latitudinal cut on the circumference of balloon assembly 40, as shown in FIG. 2.

In other embodiments, balloon assembly 40 may comprise multiple patches 50 adapted to cover multiple respective openings (such as openings 48) formed in wall 54 as described above.

Note that openings 48 are typically formed at different respective locations than electrodes 42. The distances between couples of a given TC 44 and its closest electrode 42 neighbor along outer surface 55, may be uniform or may vary among different locations on outer surface 55.

The leads for electrodes 42 (not shown) can be created in a similar manner as the thermocouple, except that the lead is left electrically exposed on the surface of the balloon and then electrode 42 is formed over the lead. Alternatively, the lead may be secured to the surface of the balloon with a conductive epoxy. The lead for electrode 42 may constitute a thermocouple where one wire is also used to deliver RF current and sense electrograms.

FIG. 3 is a flow chart that schematically illustrates a method for producing a balloon assembly of a catheter distal end, in accordance with an embodiment of the present invention. The method begins with coupling balloon assembly 40 to the jig, at a balloon coupling step 100. In some embodiments, the jig is configured to set balloon assembly 40 to an expanded position.

At a sputtering process step 102, electrodes 42 are sputtered on outer surface 55 of balloon assembly 40, during a production process in which balloon assembly 40 is coupled to the jig.

In an embodiment, the jig (shown in FIG. 4 below) may comprise a hollow mask assembly configured to contain balloon assembly 40 being fabricated. In an embodiment, the mask assembly may have one or more patterned openings through which, during the sputtering process, electrodes 42 are deposited on selected locations of assembly 40 that are exposed by the patterned openings.

In some embodiments, during sputtering process step 102, which is typically carried out under environmental vacuum conditions in a sputtering process chamber (not shown), one or more electron beams or ion beams impinge on a typically metallic target so as to sputter atoms and/or ions from the target.

In an embodiment, the target is made from gold or from any other suitable material that will be deposited on balloon assembly 40. In an embodiment, balloon assembly 40 is inflated, typically with an inert gas such as argon, before being inserted into the mask assembly. An operator mounts the mask assembly into the sputtering process chamber and pumps the air out of the process chamber so as to create a vacuum therein.

In some embodiments, there may be multiple targets made from gold, palladium, titanium-tungsten, silver, or other suitable metals to provide layers of metal to improve adhesion.

In a presence of vacuum, outer surface 55 of balloon assembly 40 inflates to press into an internal surface of the mask assembly. In this embodiment, the sputtered atoms pass through the patterned openings of the mask assembly and are deposited on balloon assembly 40 only at the intended locations on outer surface 55, so as to form electrodes 42 thereon.

At an opening formation step 104, one or more openings, such as opening 48, are formed at given locations in wall 54 of balloon assembly 40. In some embodiments, to produce the openings, wall 54 is cut along a single latitudinal of balloon assembly 40, as shown in FIG. 2 above. In other embodiments, any suitable shapes and number of cuts are formed in wall 54 by laser, heated needle, or mechanical means.

At a threading step 106, leads 46 are threaded through opening 48, such that TCs 44 and leads (not shown) for electrodes 42 are mounted on outer surface 55 of balloon assembly 40.

In some embodiments, only leads 46 are threaded through opening 48, and subsequently, each TC 44 is coupled to a respective lead 46. In other embodiments, an assembly comprising TC 44 coupled to lead 46 is threaded through opening 48.

At a patch placement step 108, one or more patches, such as patch 50, are cemented to the given locations on outer surface 55 of balloon assembly 40, so as to couple respective TCs 44 at the predefined locations on outer surface 55. In some embodiments, patches 50 are further configured to seal respective openings 48 so as to enable the inflation of balloon assembly 40.

In some embodiments, a single patch 50 may be coupled to outer surface 55 along a latitudinal cut on the circumference of balloon assembly 40, as shown in FIG. 2 above.

In other embodiments, balloon assembly 40 may comprise multiple patches 50 adapted to cover multiple respective openings formed at the given locations in wall 54 as described above.

In some embodiments, patches 50 are configured to adhere to outer surface 55, so as to block undesired exchange of fluids, through openings 48, between the internal volume of assembly 40 and blood vessel 26, or any other organ of patient 28. Following step 108, the method terminates.

In alternative embodiments, at least some of the steps of the method described above may be carried out in a different order. For example, sputtering process step 102 may be carried out after patch placement step 108.

FIG. 4 is a schematic, sectional view of a balloon assembly 65 contained within a mask assembly 60, in accordance with an embodiment of the present invention. Balloon assembly 65 may replace, for example, balloon assembly 40 of FIG. 1 above.

In the example of FIG. 4, mask assembly 60 serves as a jig for producing balloon assembly 65. In an embodiment, the configuration depicted in FIG. 4 corresponds to sputtering process step 102 described in FIG. 3 above. In some embodiments, mask assembly 60 has a substantially spherical shape and may comprise two detachable hemispheres 62 and 64. In an embodiment, the hemispheres are detached from one another during the insertion of balloon assembly 65 into mask assembly 60, and reattached to one another so as to contain assembly 40 therein.

In some embodiments, mask assembly 60 is made from metal, or any other suitable rigid material, which is adapted to withstand the vacuum applied during the sputtering process described in step 102, without its shape being deformed.

In some embodiments, balloon assembly 65 is inflated (partially or fully), typically with an inert gas 80 such as argon, before being inserted into mask assembly 60. In alternative embodiments, balloon assembly may be inflated after being inserted into mask assembly 60, or using any other suitable inflating sequence.

In an embodiment, mask assembly 60 may comprise one or more intrusions 74 that correspond with protrusions 72 of balloon assembly 65. Protrusions 72 and intrusions 74 may be used for aligning assemblies 65 and 60 to one another so as to enable accurate formation of electrodes 42 at their intended positions on assembly 65.

For example, protrusions 72 of balloon assembly 65 may serve as inflating sleeves, which are sealed at their distal ends and are substantially narrower than the maximal diameter of assembly 65 when the balloon assembly is inflated to an expanded position.

In an embodiment, intrusions 74 may be located at upper pole 76 of hemisphere 62 and at lower pole 78 of hemisphere 64. In this embodiment, protrusions 72 of assembly 65 fit into intrusions 74 of assembly 60, thereby aligning assemblies 65 and 60 to one another. In other embodiment, any suitable alternative alignment technique may be used.

In some embodiments, assembly 65 may be inflated to a degree that leaves (after being inserted into assembly 60) a spacing 70 (filled with air) between assemblies 65 and 60. In some embodiments, a production operator of assembly 65 may use spacing 70 to fine-tune the alignment between assemblies 65 and 60.

In some embodiments, hemisphere 62 comprises one or more openings 68 patterned between bars 75. Sputtering process step 102, which is typically carried out in vacuum, causes the deformable external surface of balloon assembly 65 to attach to the internal surface of mask assembly 60.

In an embodiment, assemblies 65 and 60 are attached to one another, so that the sputtered atoms pass through openings 68 of assembly 60 and are deposited on assembly 65 only at the intended positions on the external surface of assembly 65, so as to form electrodes 42 thereon.

In the example of FIG. 4, hemisphere 64 has a solid profile (i.e., without openings) so that after sputtering process 102, the external surface of assembly 65 that is located under bars 75 and under hemisphere 64, is not coated with metal during sputtering process 102.

In some embodiments, at opening formation step 104 of FIG. 3 above, opening 48 may be formed in assembly 65 at the locations under bars 75 and/or under hemisphere 64, which are not coated by the metal layer of electrodes 42. Furthermore, patches 50 and TCs 44 may be coupled to the external surface of assembly 65 at location not coated with metal, such as the locations under bars 75 and/or under hemisphere 64.

The examples of FIGS. 1-4 refer to a specific configurations of balloon assemblies 40 and 65 and to a specific configuration of mask assembly 60. These configurations, however, are chosen purely for the sake of conceptual clarity. In other embodiments, hemisphere 64 may have openings, and hemisphere 62 may have any suitable patterned openings, different than openings 68 shown in FIG. 4.

In alternative embodiments, the disclosed techniques can be used, mutatis mutandis, in various other types of distal end assemblies and balloon catheter.

Furthermore, the description of the jig is given purely by way of example. In alternative embodiments, balloon assemblies 40 and/or 65 may be coupled to any other suitable jig or mounted on any other type of manufacturing tool.

Although the embodiments described herein mainly address cardiology procedures, the methods and systems described herein can also be used in other applications, such as otolaryngology or neurology procedures.

It will thus be appreciated that the embodiments described above are cited by way of example, and that the present invention is not limited to what has been particularly shown and described hereinabove. Rather, the scope of the present invention includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A method for producing a medical instrument, the method comprising:
coupling a balloon-based distal end of the medical instrument to a jig that sets the distal end to an expanded position; and
while the distal end is coupled to the jig:
depositing one or more electrodes on an outer surface of the distal end;
forming one or more openings in a wall of the distal end, and threading through the openings respective leads coupled to at least one of respective sensors and electrodes that are mounted on the outer surface of the distal end; and
coupling, on the outer surface of the distal end, one or more patches that cover the openings and couple the at least one of respective sensors and electrodes to the outer surface of the distal end.

2. The method according to claim 1, wherein forming the openings comprises cutting a latitudinal opening in the wall of the balloon-based distal end.

3. The method according to claim 1, wherein the sensors comprise one or more thermocouples (TCs).

4. The method according to claim 1, wherein coupling the balloon-based distal end to the jig comprises inserting the balloon-based distal end into a hollow templates having one or more patterned openings.

5. The method according to claim 4, wherein depositing the electrodes comprises sputtering atoms or ions through the patterned openings.

6. The method according to claim 5, wherein sputtering the atoms or ions comprises impinging electrons or ions on a sputtering target.

7. The method according to claim 5, and comprising, before depositing the electrodes through the patterned openings, attaching the outer surface of the balloon-based distal ends to an inner surface of the hollow template, by creating vacuum around the balloon-based distal ends.

8. The method according to claim 1, wherein the balloon-based distal end comprises an inflatable balloon made from polyethylene terephthalate (PET).

9. The method according to claim 1, wherein the balloon-based distal end comprises an inflatable balloon made from polyurethane.

10. The method according to claim 1, wherein the balloon-based distal end comprises an inflatable balloon made from polyether block amide.

11. The method according to claim 1, wherein coupling the one or more patches that cover the openings comprises sealing the openings.

12. The method according to claim 1, wherein coupling the one or more patches comprises cementing the at least one of respective sensors and electrodes to the outer surface of the distal end.

13. The method according to claim 1, wherein the electrodes comprise one or more ablation electrodes.

14. The method according to claim 1, wherein the sensors comprise one or more electrophysiology (EP) sensing electrodes.
